(19) United States
(12) Patent Application Publication
(10) Pub. No.: US 2004/0097599 A1
(43) Pub. Date: May 20, 2004

(54) Antibacterial and antifungal compositions based on branched C-11 alcohols

(76) Inventors: Tiziana Rodriguez, Monte Isola (IT); Calogero Genova, Vizzolo Predabissi-Milan (IT); Luigi Valtorta, Desio-Milan (IT); Agostino Zatta, San Giuliano Milanese-Milan (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/469,226

(22) PCT Filed: Feb. 22, 2002
(86) PCT No.: PCT/EP02/01977

(30) Foreign Application Priority Data
Mar. 8, 2001 (IT) M12001A000477

(51) Int. Cl. A61K 31/045

(52) U.S. Cl. 514/724

(57) ABSTRACT

Antibacterial and antigungine compositions which comprise a carrier base and at least one branched alcohol containing (11) carbon atoms in a concentration higher than 0.0005% by weight.

The present invention relates to antibacterial compositions based on long-chain branched alcohols.

More specifically, the present invention relates to antibacterial compositions also having an anti-mold and anti-yeast activity based on branched alcohols with 11 carbon atoms.

Even more specifically, the present invention relates to the use of branched alcohols having 11 carbon atoms, optionally etherified, as antibacterial and antifungine agents.

The use of long-chain alcohols with an antibacterial function, is known. For example, the “Journal of Agricultural and Food Chemistry”, 1993, 41, 2447-2450, describes the antibacterial activity of long-chain linear C₆-C₂₀ alcohols towards Streptococcus mutans. According to what is specified in this scientific article, 1-dodecanol and 1-tridecanol alcohols have proved to be particularly active, with a MIC (Minimum Inhibitory Concentration) equal to 6.25 μg/ml.

In “Biorganic & Medicine Chemistry Letters”, 1993,3, 6, 1305-1308, the antibacterial activity is described of linear C₈-C₁₂ alcohols with respect to Staphylococcus aureus and linear C₇-C₁₆ alcohols with respect to Propionibacterium acnes.

Other articles appearing in scientific magazines, such as “Journal of Antibacterial and Antifungine Agents”, 1980, 8, 8, 1-7 and “Journal of Pharmaceutical Science”, 1980, 69, 4, 381, describe not only the antibacterial activity of n-alkanols but also their activity against mold and yeast.

In particular, international patent application WO 99/51093 describes the use of 1-nonanol, 1-decanol or 1-undecanol, or their mixtures, as base agents for preparing compositions having an antibacterial activity towards a wide range of bacteria and yeast.

Scientific literature therefore provides ample documentation on the use of long-chain linear alcohols as antibacterial agents or with anti-mold/yeast functions whereas, as far as alcohols with a branched structure are concerned, the same literature does not give sufficient information to allow their evaluation. The experimental evidence provided hereunder, demonstrates that long-chain branched alcohols generally do not have particularly interesting antibacterial and antifungine properties.

The Applicant however has surprisingly found that branched C₁₁ alcohols, also available on the market under the trade-name of ISALCHEM 11, owned by the Applicant, have proved to be very active against both gram-positive and gram-negative bacteria and also against mycetes such as mold and yeast, thus showing counter-tendency behaviour with respect to that of other long-chain branched alcohols. Analogous results have been obtained by etherifying C₁₁ branched alcohols with oxides such as ethylene oxide and/or propylene oxide.

An object of the present invention therefore relates to antibacterial and antifungine compositions which comprise a carrier base and at least one branched alcohol containing 11 carbon atoms, optionally etherified, in a concentration higher than 0.0005% by weight.

The branched alcohol is preferably present in the antibacterial or antifungine compositions, object of the present invention, in concentrations ranging from 0.001 to 1% by weight. These compositions can be used alone or combined with other conventional additives used for these purposes or with other substances having antibacterial and/or antifungine properties compatible with C₁₁ alcohol.

Examples of branched alcohols containing 11 carbon atoms are 2-methyl-1-decanol, 2-ethyl-1-nonanol, 2-propyl-1-octanol and 2-butyl-1-heptanol. In particular, mixtures of branched C₁₁ alcohols are preferred, which comprise:

a) 30-50% by weight of 2-methyl-1-decanol;

b) 15-25% by weight of 2-ethyl-1-nonanol;

c) 15-25% by weight of 2-propyl-1-octanol; and

d) 15-25% by weight of 2-butyl-1-heptanol.

A preferred branched alcohol containing 11 carbon atoms according to the present invention is that known under the trade-name of ISALCHEM 11 which substantially has the following composition: 2-methyl-1-decanol=39% by weight; 2-ethyl-1-nonanol=20% by weight; 2-propyl-1-octanol=19% by weight; 2-butyl-1-heptanol=20% by weight and about 2% by weight of 1-undecanol and/or alcohols having a lower or higher molecular weight. ISALCHEM 11 is particularly preferred for its antibacterial activity against Staphylococcus aureus and against Proteus mirabilis and for its contemporary antifungine activity towards Candida albicans yeast and Penicillium notatum and Aspergillus niger mold.

The means for carrying the branched C₁₁ alcohol are those traditionally used for carrying long-chain linear alcohols such as C₁-C₄ alcohols, water/oil multiphase compositions, surface-active agents such as polysorbate, etc. Further examples of carrier materials are available in international patent application WO 99/51093.

A further object of the present invention relates to the use of branched C₁₁ alcohols, optionally mixed with linear C₈-C₂₀ alcohols or with branched C₁₂-C₁₅ alcohols, as antibacterial and antifungine agents. The use of branched C₁₁ alcohol is such as to give an MIC value equal to or less than 1% by weight whereas the other long-chain alcohols are used in a weight ratio ranging from 0 to 1 with respect to the C₁₁ alcohol.

The branched C₁₁ alcohols according to the present invention can also be used in etherified form, for example with 1-5 moles, preferably 1-3 moles, of ethylene oxide and/or propylene oxide.

The branched alcohol ISALCHEM 11 was used, for illustrative but non-limiting purposes, in a MIC test. The results were compared with analogous branched products having a longer chain and commercialized by the Applicant under the names of ISALCHEM 12 (branched alcohol with 12 carbon atoms), ISALCHEM 13 (branched alcohol with 13 carbon atoms), ISALCHEM 14 (branched alcohol with 14 carbon atoms), ISALCHEM 15 (branched alcohol with 15 carbon atoms) and ISALCHEM 123 (branched alcohol containing 12-13 carbon atoms). The results obtained are indicated in the following table.

TABLE

| | BACTERIA | | MYCETES | | |
|---|---|---|---|---|---|
| | GRAM + | | YEAST | MOLD | MOLD |
| | Staphylococcus | GRAM − | Candida | Aspergillus | Penicillium |
| | aureus | Proteus mirabilis | albicans | niger | notatum |
| ISALCHEM 11 | 78 ppm | 5000 ppm | 625 ppm | 625 ppm | 10 ppm |
| ISALCHEM 12 | 0.5% | >1% | >1% | >1% | 5 ppm |
| ISALCHEM 13 | >1% | >1% | >1% | >1% | 39 ppm |
| ISALCHEM 14 | >1% | >1% | >1% | >1% | 1250 ppm |
| ISALCHEM 15 | >1% | >1% | >1% | >1% | 1250 ppm |
| ISALCHEM 123 | >1% | >1% | >1% | >1% | 20 ppm |

1. Antibacterial and antifungine compositions which comprise a carrier base and at least one branched alcohol containing 11 carbon atoms, optionally etherified, in a concentration higher than 0.0005% by weight.

2. The compositions according to claim 1, wherein the branched alcohol is present in concentrations ranging from 0.001 to 1% by weight.

3. The compositions according to claim 1 or 2, wherein the branched alcohols containing 11 carbon atoms are selected from 2-methyl-1-decanol, 2-ethyl-1-nonanol, 2-propyl-1-octanol and 2-butyl-1-heptanol.

4. The compositions according to any of the previous claims, wherein the mixtures of branched C₁₁ alcohols comprise:
a) 30-50% by weight of 2-methyl-1-decanol;
b) 15-25% by weight of 2-ethyl-1-nonanol;
c) 15-25% by weight of 2-propyl-1-octanol; and
d) 15-25% by weight of 2-butyl-1-heptanol.

5. The compositions according to claim 4, wherein the branched alcohol containing 11 carbon atoms essentially consists of: 2-methyl-1-decanol =39% by weight; 2-ethyl-1-nonanol=20% by weight; 2-propyl-1-octanol=19% by weight; 2-butyl-1-heptanol=20% by weight and about 2% by weight of 1-undecanol and/or alcohols having a lower or higher molecular weight.

6. The compositions according to any of the previous claims, wherein the means for carrying the branched C₁₁ alcohol are selected from C₁-C₄ alcohols, water/oil multiphase compositions and surface-active agents such as polysorbate.

7. The compositions according to any of the previous claims wherein the branched C₁₁ alcohols are etherified with 1-5 moles of ethylene oxide and/or propylene oxide.

8. The use of branched C₁₁ alcohols, optionally mixed with linear C₈-C₂₀ alcohols or with branched C₁₂-C₁₅ alcohols, as antibacterial and antifungine agents.

9. The use according to claim 7, wherein the concentration of the branched C₁₁ alcohol is such as to give a MIC (Minimum Inhibitory Concentration) value equal to or lower than 1% by weight whereas the other long-chain alcohols are used in a weight ratio ranging from 0 to 1 with respect to the C₁₁ alcohol.

10. The use according to claim 8 or 9, wherein the branched C₁₁ alcohols are etherified with 1-5 moles of ethylene oxide and/or propylene oxide.
